(19) United States
(12) Patent Application Publication
(10) Pub. No.: US 2025/0312096 A1
(43) Pub. Date: Oct. 9, 2025

(54) VENOUS INTERVENTIONAL INTEGRATED DEVICE FOR BRAIN TISSUE BIOPSY OR ELECTRODE PLACEMENT VIA CEREBRAL SURFACE VEINS

(71) Applicant: Second Affiliated Clinical College of Fujian University of Traditional Chinese Medicine, Fuzhou (CN)

(72) Inventors: Chenghan WU, Fuzhou (CN); Min LIN, Fuzhou (CN); Sheng LIN, Fuzhou (CN); Jing CHEN, Fuzhou (CN); Lili GAO, Fuzhou (CN); Yuansheng LIAO, Fuzhou (CN); Ruoya WU, Fuzhou (CN)

(73) Assignee: Second Affiliated Clinical College of Fujian University of Traditional Chinese Medicine, Fuzhou (CN)

(21) Appl. No.: 19/243,721

(22) Filed: Jun. 20, 2025

Related U.S. Application Data
(63) Continuation of application No. PCT/CN2024/081187, filed on Mar. 12, 2024.

(30) Foreign Application Priority Data
Mar. 1, 2024 (CN) 202410237895.4

(51) Int. Cl. A61B 18/14

(52) U.S. Cl. CPC A61B 18/1492

(57) ABSTRACT

A venous interventional integrated device for a brain tissue biopsy or a brain-computer interface electrode placement via cerebral surface veins includes a catheter and a deformable balloon, the catheter is connected to the proximal end of the balloon, the catheter includes at least three channels: a balloon inflation channel, a guide channel, and a working channel, the balloon inflation channel is connected to the balloon lumen to inflate the balloon by injecting contrast agent, the guide channel passes through the balloon lumen from the proximal end of the balloon and exits from the distal end of the balloon for threading a micro-guidewire, the working channel extends from the sidewall of the catheter at a set distance from the proximal end of the balloon, and a puncture needle is threaded through the working channel and configured for biopsy sampling or electrode placement on the surface or deep within the brain tissue.

### CROSS REFERENCE TO THE RELATED APPLICATIONS

This application is a continuation application of International Application No. PCT/CN2024/081187, filed on Mar. 12, 2024, which is based upon and claims priority to Chinese Patent Application No. 202410237895.4, filed on Mar. 1, 2024, the entire contents of which are incorporated herein by reference.

### TECHNICAL FIELD

The invention relates to the field of medical devices, specifically to a venous interventional integrated device for a brain tissue biopsy or a brain-computer interface electrode placement via cerebral surface veins.

### BACKGROUND

Currently, brain tissue biopsy or placement of electrodes on the surface or deep within the brain is performed using craniotomy or drilling methods; traditional methods cause significant damage to the body and brain tissue, are prone to bleeding, cause considerable patient suffering, are complex to operate, have poor positioning accuracy, and are prone to deviations in biopsy sampling or brain-computer interface electrode placement.

### SUMMARY

The purpose of the invention is to provide a venous interventional integrated device for a brain tissue biopsy or a brain-computer interface electrode placement via cerebral surface veins, which is multifunctional, allowed to be used for both biopsy and electrode placement, with accurate positioning, simple operation, high success rate, and minimal environmental impact during operation.

To achieve the above purpose, the technical solution adopted by the invention is: a venous interventional integrated device for a brain tissue biopsy or a brain-computer interface electrode placement via cerebral surface veins, including a catheter and a balloon, wherein the balloon is deformable, the catheter is connected to a proximal end of the balloon, and the catheter includes at least three channels, wherein the at least three channels include a balloon inflation channel, a guide channel, and a working channel; the balloon inflation channel is connected to a balloon lumen to inflate the balloon by injecting a contrast agent, the guide channel passes through the balloon lumen from the proximal end of the balloon and exits from a distal end of the balloon for threading a micro-guidewire, the working channel extends from a sidewall of the catheter at a set distance from the proximal end of the balloon, and a puncture needle is threaded through the working channel, the puncture needle exits from the distal end of the working channel for a biopsy sampling or an electrode placement.

Further, the puncture needle has an internal channel and a front opening, a front end of the puncture needle penetrates a target object; a puncture depth of the puncture needle is allowed to be adjusted as needed; for the biopsy sampling, a sampling hook is delivered through the internal channel to the front end of the puncture needle to sample the target object; for the brain-computer interface electrode placement, an electrode is delivered through the internal channel to the front end of the puncture needle and placed into the target object.

Further, the balloon and the catheter are threaded over the micro-guidewire through the guide channel and guided to the target position by the micro-guidewire during an operation.

Further, the catheter includes a plurality of working channels, each extending from different sides of the catheter, allowing a selection of an appropriate direction for the biopsy sampling or the electrode placement during an operation.

Further, except for the balloon inflation channel being connected to the balloon lumen, the guide channel and working channel are isolated from the balloon lumen.

Further, the balloon inflation channel, the guide channel, and the working channel are integrated into a single catheter, with each channel separated at the proximal end of the catheter for connecting to a contrast agent injection device, threading the micro-guidewire, and threading the puncture needle, respectively.

Further, the distal end and the proximal end of the balloon and an exit end of the working channel of the catheter are each provided with metal markers to facilitate positioning of the balloon and an exit position of the puncture needle during an operation.

Further, the venous interventional integrated device is equipped with a sealing and repair wire, the sealing and repair wire is allowed to be delivered through the working channel, a front end of the sealing and repair wire is provided with a sealing head, wherein the sealing head is severable, a front end of the sealing head is provided with a self-expandable umbrella-shaped biological membrane made of an absorbable material; when the sealing and repair wire is delivered through the working channel, the umbrella-shaped biological membrane is constrained by the working channel and folded around a periphery of the sealing head, and when the umbrella-shaped biological membrane exits the working channel, the umbrella-shaped biological membrane automatically expands forward and outward.

Further, the venous interventional integrated device is equipped with a sealing and repair wire, the sealing and repair wire is allowed to be delivered through the working channel, a front end of the sealing and repair wire is provided with a sealing head, wherein the sealing head is severable, a front end and a rear end of the sealing head are provided with two umbrella-shaped biological membranes made of an absorbable material, wherein the two umbrella-shaped biological membranes are self-expandable and arranged in opposite directions; when the sealing and repair wire is delivered through the working channel, the two umbrella-shaped biological membranes are constrained by the working channel and folded around the front end and the rear end of the sealing head, and when a front umbrella-shaped biological membrane exits the working channel, the front umbrella-shaped biological membrane automatically expands forward and outward, and when a rear umbrella-shaped biological membrane exits the working channel, the rear umbrella-shaped biological membrane automatically expands backward and outward.

Further, the working channel is further configured to deliver a venous wall repair material to seal and repair a puncture site, the venous wall repair material is made of a biological material, wherein the biological material is adhesive to a venous wall and allowed to be absorbed by the venous wall.

Compared with the existing technology, the invention has the following beneficial effects: the invention integrates the balloon, catheter, biopsy device, and electrode placement device into an integrated device, which has a multi-channel design, the working channel can be used for both biopsy and placement of various electrodes such as human-computer dialogue electrodes, brain pacemaker electrodes, neural regulation electrodes, and brain function localization electrodes, while the balloon inflation channel is connected to the balloon, by expanding the balloon to reversibly block venous blood flow, the working position is not affected by blood flow, achieving the advantages of minimal trauma, convenience, accurate positioning, and flexible deployment. Therefore, the invention has strong practicality and broad application prospects.

### BRIEF DESCRIPTION OF THE DRAWINGS

FIG. 1 is a schematic structural diagram of the venous interventional integrated device according to an embodiment of the invention;

FIG. 2 is a schematic diagram of the first working state of the venous interventional integrated device according to an embodiment of the invention;

FIG. 3 is a schematic diagram of the second working state of the venous interventional integrated device according to an embodiment of the invention;

FIG. 4 is a partial enlarged view of the puncture needle in FIG. 2;

FIG. 5 is a schematic diagram of the working state of the puncture needle for electrode placement according to an embodiment of the invention;

FIG. 6 is a schematic diagram of the first working state of the sealing and repair wire in the first embodiment of the invention;

FIG. 7 is a schematic diagram of the second working state of the sealing and repair wire in the first embodiment of the invention;

FIG. 8 is a schematic diagram of the third working state of the sealing and repair wire in the first embodiment of the invention;

FIG. 9 is a schematic diagram of the fourth working state of the sealing and repair wire in the first embodiment of the invention;

FIG. 10 is a schematic diagram of the fifth working state of the sealing and repair wire in the first embodiment of the invention;

FIG. 11 is a schematic diagram of the first working state of the sealing and repair wire in the second embodiment of the invention;

FIG. 12 is a schematic diagram of the second working state of the sealing and repair wire in the second embodiment of the invention;

FIG. 13 is a schematic diagram of the third working state of the sealing and repair wire in the second embodiment of the invention;

FIG. 14 is a schematic diagram of the fourth working state of the sealing and repair wire in the second embodiment of the invention;

FIG. 15 is a schematic diagram of the fifth working state of the sealing and repair wire in the second embodiment of the invention;

FIG. 16 is a schematic diagram of the sixth working state of the sealing and repair wire in the second embodiment of the invention.

### DETAILED DESCRIPTION OF THE EMBODIMENTS

The following describes the invention in detail with reference to the drawings and embodiments.

It should be noted that the following detailed description is exemplary and is intended to provide further explanation of the application. Unless otherwise specified, all technical and scientific terms used herein have the same meaning as commonly understood by one of ordinary skill in the art to which this application belongs.

It should be noted that the terminology used herein is for the purpose of describing specific embodiments and is not intended to limit the exemplary embodiments of the application. As used herein, unless the context clearly indicates otherwise, the singular forms are intended to include the plural forms, and it should also be understood that when the terms “comprise” and/or “include” are used in this specification, they specify the presence of stated features, steps, operations, devices, components, and/or combinations thereof.

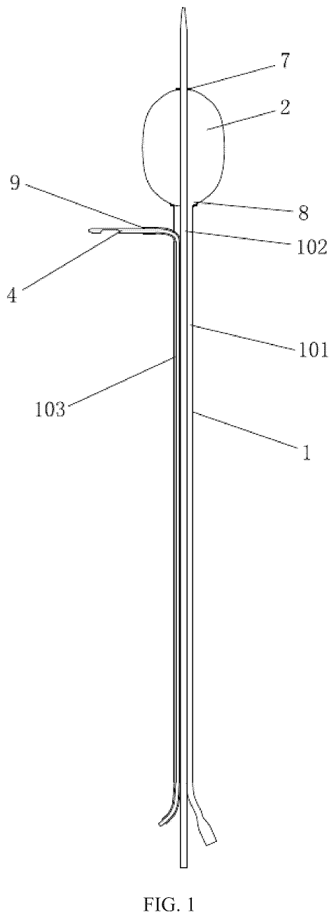

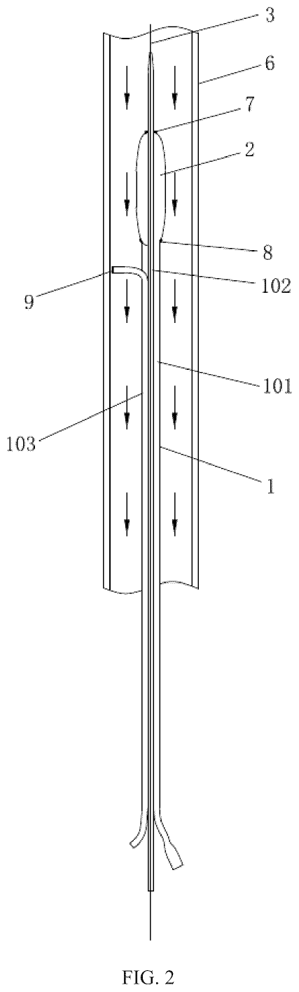

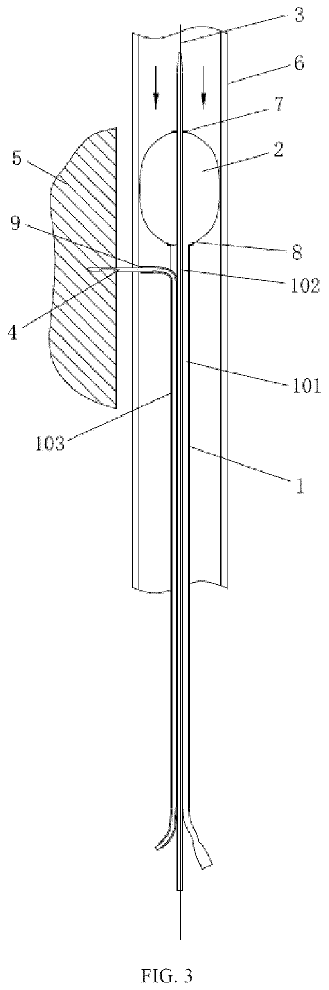

As shown in FIGS. 1-3, this embodiment provides a venous interventional integrated device for a brain tissue biopsy or a electrode placement via cerebral surface veins, including a catheter 1 and a deformable balloon 2, the catheter 1 is connected to a proximal end of the balloon 2, the catheter 1 includes at least three channels, wherein the at least three channels include a balloon inflation channel 101, a guide channel 102, and a working channel 103; the balloon inflation channel 101 is connected to a balloon lumen to inflate the balloon by injecting a contrast agent, the guide channel 102 passes through the balloon lumen from the proximal end of the balloon and exits from a distal end of the balloon for threading a micro-guidewire 3, the working channel 103 extends from a sidewall of the catheter at a set distance from the proximal end of the balloon, and a puncture needle 4 is threaded through the working channel, the puncture needle 4 exits from the distal end of the working channel 103 for a biopsy sampling or an electrode placement on the surface or deep within the brain tissue.

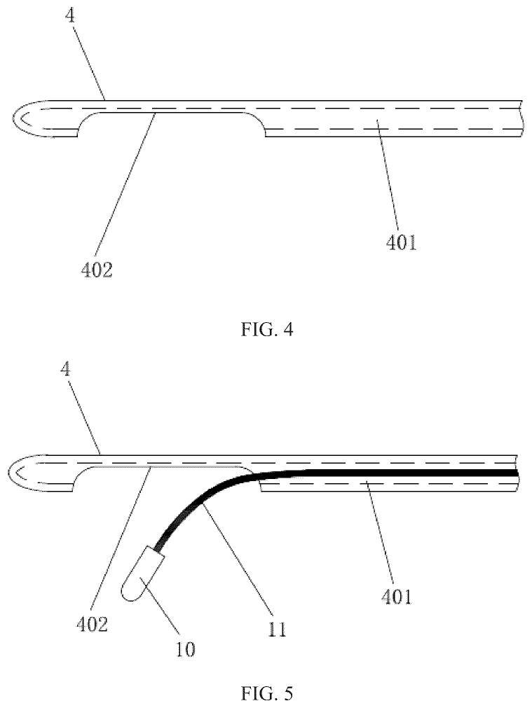

For biopsy sampling or electrode placement, as shown in FIG. 4, the puncture needle 4 has an internal channel 401 and a front opening 402, a front end of the puncture needle penetrates a target object 5. For biopsy sampling, a sampling hook is delivered through the internal channel to the front end of the puncture needle to sample the target object. For electrode placement, as shown in FIG. 5, an electrode 10 is delivered through a guidewire 11 along the internal channel to the front end of the puncture needle, and the electrode is placed into the target object.

The balloon 2 and the catheter 1 are threaded over the micro-guidewire 3 through the guide channel and guided to the target position in the blood vessel 6, i.e., the position for biopsy sampling or electrode placement.

In this venous interventional integrated device, except for the balloon inflation channel 101 being connected to the balloon 2 lumen, the guide channel 102 and working channel 103 are isolated from the balloon 2 lumen and the balloon inflation channel 101, i.e., the connection between the guide channel 102 and the balloon is sealed, and the working channel 103 does not penetrate the balloon, ensuring that each channel can work independently without mutual interference. The balloon inflation channel 101, the guide channel 102, and the working channel 103 are integrated into a single catheter 1, the distal end of the catheter 1 is connected to the proximal end of the balloon 2, and the proximal end of the catheter 1 separates into each channel for connecting to a contrast agent injection device, threading the micro-guidewire 3, and the threading a puncture needle 4, respectively.

In this embodiment, the distal end and the proximal end of the balloon 2 and an exit end of the working channel 103 of the catheter 1 are each provided with metal markers 7, 8, 9 to facilitate positioning of the balloon 2 and an exit position of the puncture needle 4 during an operation.

To avoid the need to adjust the exit direction of the puncture needle during operation, or to facilitate biopsy sampling or electrode placement in different orientations, in a preferred embodiment of the invention, the catheter includes a plurality of working channels, each extending from different sides of the catheter, allowing a selection of an appropriate direction for the biopsy sampling or the electrode placement during an operation.

The method of using the venous interventional integrated device for the brain tissue biopsy or the electrode placement via the cerebral surface veins provided in this embodiment is as follows:

Thread the balloon and catheter over the micro-guidewire through the guide channel.

Continuously advance the balloon and catheter distally until the balloon and catheter are guided to the target position by the micro-guidewire.

Adjust the orientation of the exit end of the working channel to face the target object.

Inject contrast agent into the balloon through the balloon inflation channel to expand the balloon and block the blood vessel, preventing blood flow from affecting the biopsy sampling or electrode placement.

Deliver the puncture needle to the exit end of the working channel, exit from the working channel, and penetrate the target object. If biopsy sampling is to be performed, deliver a sampling hook through the internal channel of the puncture needle to the front end of the puncture needle and sample the target object. If electrode placement is to be performed, deliver an electrode through the internal channel of the puncture needle to the front end of the puncture needle and place the electrode into the target object.

The venous interventional integrated device for the brain tissue biopsy or the electrode placement via the cerebral surface veins of the invention also provides multiple solutions for repairing the puncture site (venous wall) after completing the task.

In one embodiment of the invention, the venous interventional integrated device is equipped with a sealing and repair wire 12 that allowed to be delivered through the working channel, the front end of the sealing and repair wire 12 is provided with a severable sealing head 13, the front end of the sealing head 13 is provided with a self-expandable umbrella-shaped biological membrane 14 made of absorbable material; when the sealing and repair wire is delivered through the working channel, the umbrella-shaped biological membrane is constrained by the working channel and folded around the periphery of the sealing head, and when the umbrella-shaped biological membrane exits the working channel, it automatically expands forward and outward.

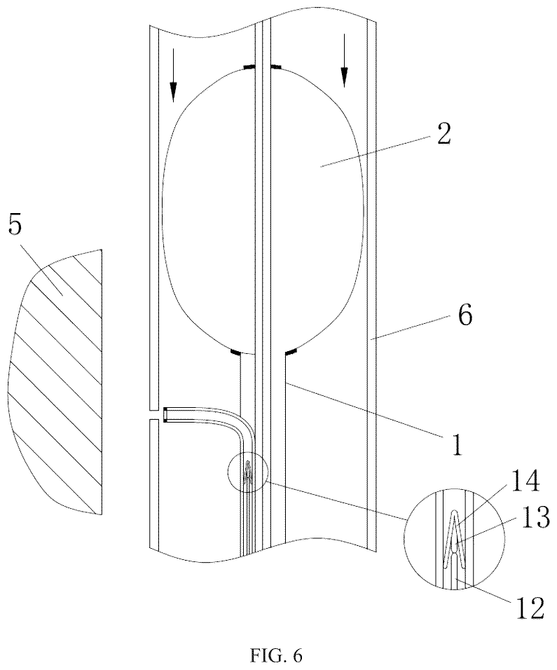

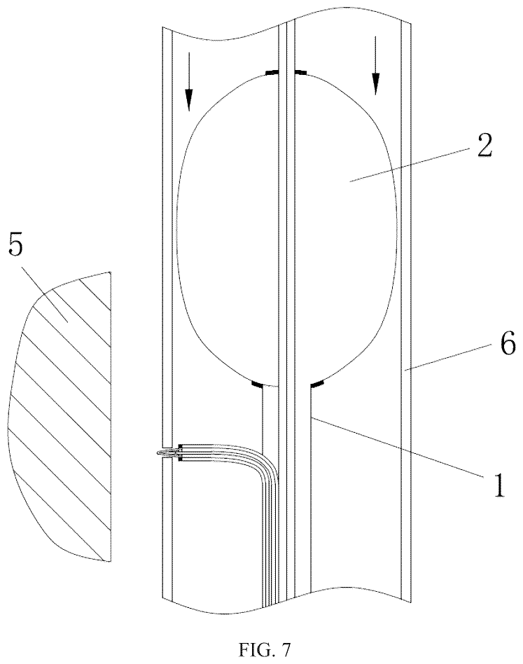

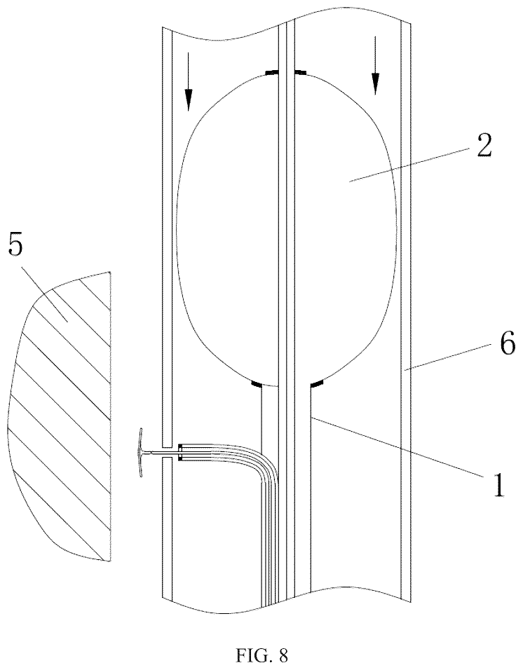

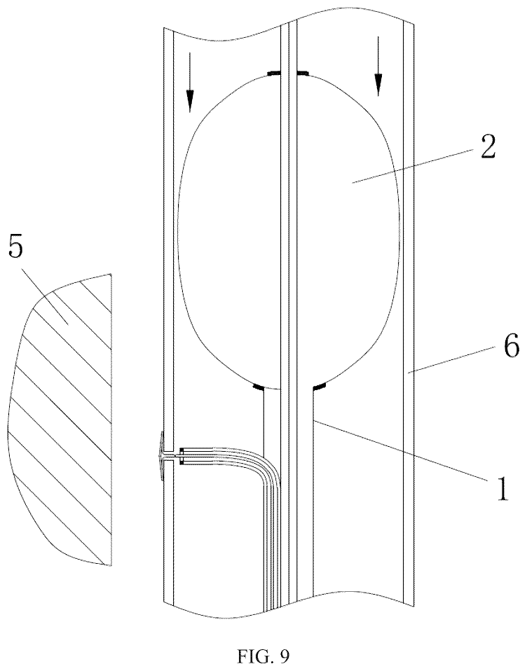

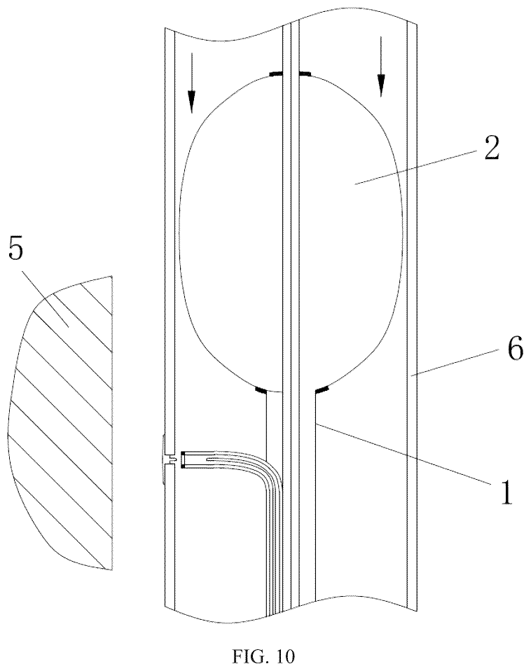

In this embodiment, when biopsy or electrode placement tasks are completed, retract the puncture needle, deliver the sealing and repair wire through the working channel towards the exit end of the working channel, as shown in FIG. 6. During delivery, the umbrella-shaped biological membrane is constrained by the working channel and folded around the periphery of the sealing head, as shown in FIGS. 6-7. When the sealing head exits the working channel, as shown in FIG. 8, the umbrella-shaped biological membrane is no longer constrained by the working channel and automatically expands. At this point, retract the sealing and repair wire to adhere the umbrella-shaped biological membrane to the outer wall of the puncture site, as shown in FIG. 9. Then, sever the sealing head, which integrates with the umbrella-shaped biological membrane to seal the puncture site, and retract the remaining part of the sealing and repair wire, as shown in FIG. 10. At this point, the contrast agent can be withdrawn, allowing the balloon to contract and restore blood flow.

In another embodiment of the invention, the venous interventional integrated device is equipped with a sealing and repair wire 15 that allowed to be delivered through the working channel. The front end of the sealing and repair wire 15 is provided with a severable sealing head 16. The front end and the rear end of the sealing head 16 are provided with two self-expandable umbrella-shaped biological membranes 17 and 18, made of an absorbable material, arranged in opposite directions. When the sealing and repair wire is delivered through the working channel, the two umbrella-shaped biological membranes are constrained by the working channel and folded around the front end and the rear end of the sealing head. When a front umbrella-shaped biological membrane exits the working channel, it automatically expands forward and outward, and when a rear umbrella-shaped biological membrane exits the working channel, it automatically expands backward and outward.

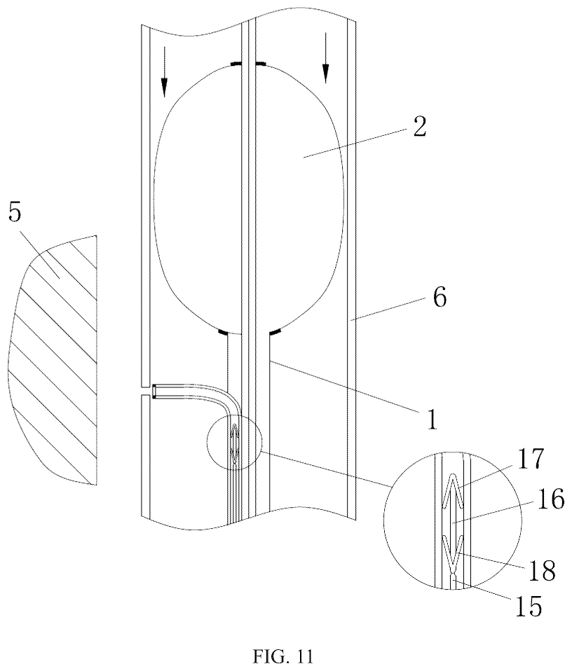

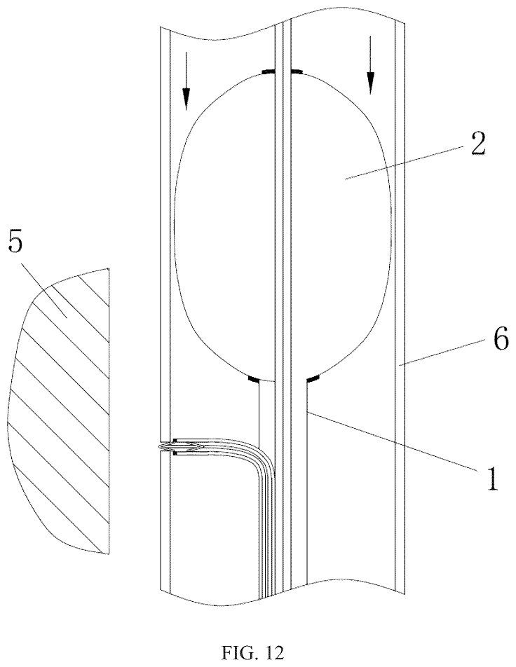

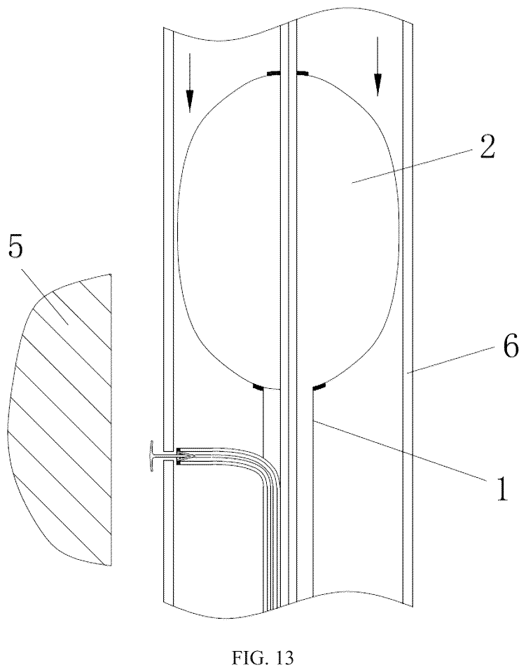

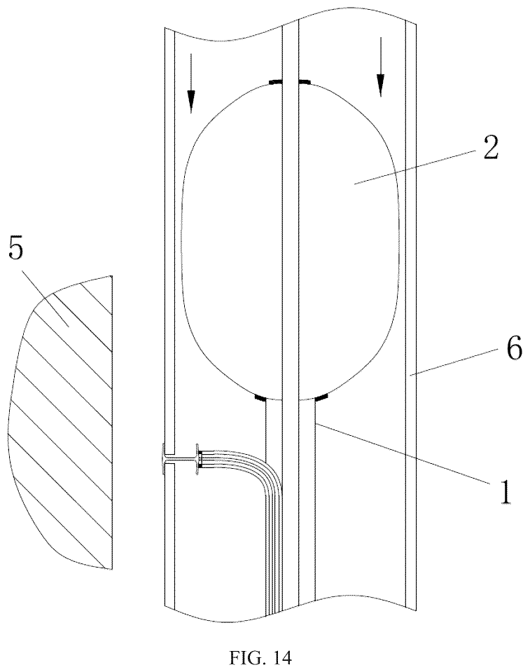

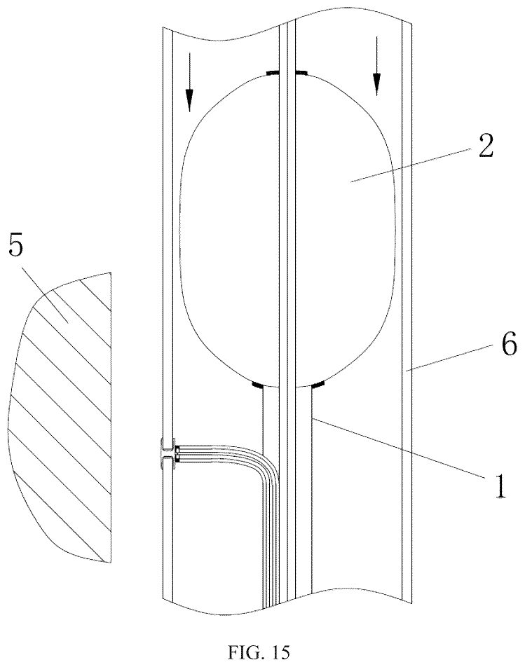

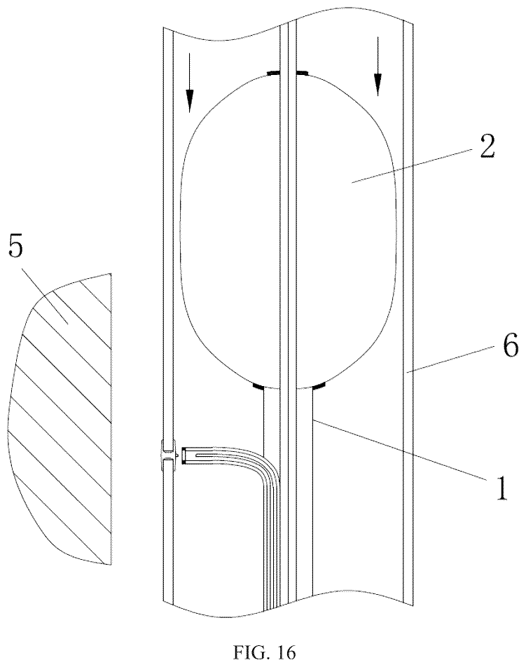

In this embodiment, after completing the biopsy or electrode placement tasks, the puncture needle is retracted, and the sealing and repair wire is delivered through the working channel towards the exit end of the working channel, as shown in FIG. 11. During delivery, the umbrella-shaped biological membranes are constrained by the working channel and folded around a periphery of the sealing head, as shown in FIGS. 11-12. When the sealing head exits the working channel, as shown in FIG. 13, the front umbrella-shaped biological membrane is no longer constrained by the working channel and automatically expands forward and outward. At this point, the sealing and repair wire is retracted to adhere the front umbrella-shaped biological membrane to the outer wall of the puncture site. Then, the working channel is slightly retracted to allow the rear umbrella-shaped biological membrane to exit the working channel, at which point it automatically expands backward and outward, as shown in FIG. 14. Next, the front end of the working channel pushes the rear umbrella-shaped biological membrane, which has expanded backward, against the inner wall of the puncture site, thereby sealing the puncture site with both forward and backward double-layer umbrella-shaped biological membranes, as shown in FIG. 15. Then, the sealing head is severed, and the sealing head integrates with the forward and backward double-layer umbrella-shaped biological membranes to seal the puncture site. The remaining part of the sealing and repair wire is retracted, as shown in FIG. 16. At this point, the contrast agent allowed to be withdrawn, allowing the balloon to continue contracting and restoring blood flow.

In yet another embodiment of the invention, the working channel is further configured to deliver and spray a venous wall repair material to seal and repair a puncture site. The venous wall repair material is made of a biological material, wherein the biological material is adhesive to a venous wall and allowed to be absorbed by the venous wall. After completing the tasks, the puncture needle is retracted, and the venous wall repair material is delivered through the working channel and sprayed from the distal end of the working channel. The sprayed venous wall repair material adheres to the puncture site, achieving the purpose of repairing the damaged venous wall.

The above descriptions are only preferred embodiments of the invention and are not intended to limit the invention in any other form. Any person skilled in the art may use the disclosed technical content to make modifications or changes to equivalent embodiments. However, any simple modifications, equivalent changes, or adaptations made to the above embodiments based on the essence of the technical solution of the invention, without departing from the content of the technical solution of the invention, shall still fall within the scope of protection of the technical solution of the invention.

What is claimed is:

1. A venous interventional integrated device for a brain tissue biopsy or a brain-computer interface electrode placement via cerebral surface veins, comprising a catheter and a balloon, wherein the balloon is deformable, the catheter is connected to a proximal end of the balloon, and the catheter comprises at least three channels, wherein the at least three channels comprise a balloon inflation channel, a guide channel, and a working channel; the balloon inflation channel is connected to a balloon lumen to inflate the balloon by injecting a contrast agent, the guide channel passes through the balloon lumen from the proximal end of the balloon and exits from a distal end of the balloon for threading a micro-guidewire, the working channel extends from a sidewall of the catheter at a set distance from the proximal end of the balloon, and a puncture needle is threaded through the working channel, the puncture needle exits from the distal end of the working channel for a biopsy sampling or an electrode placement.

2. The venous interventional integrated device for the brain tissue biopsy or the brain-computer interface electrode placement via the cerebral surface veins according to claim 1, wherein the puncture needle has an internal channel and a front opening, a front end of the puncture needle penetrates a target object; a puncture depth of the puncture needle is allowed to be adjusted as needed; for the biopsy sampling, a sampling hook is delivered through the internal channel to the front end of the puncture needle to sample the target object; for the brain-computer interface electrode placement, an electrode is delivered through the internal channel to the front end of the puncture needle and placed into the target object.

3. The venous interventional integrated device for the brain tissue biopsy or the brain-computer interface electrode placement via the cerebral surface veins according to claim 1, wherein the balloon and the catheter are threaded over the micro-guidewire through the guide channel and guided to the target position by the micro-guidewire during an operation.

4. The venous interventional integrated device for the brain tissue biopsy or the brain-computer interface electrode placement via the cerebral surface veins according to claim 1, wherein the catheter comprises a plurality of working channels, each extending from different sides of the catheter, allowing a selection of an appropriate direction for the biopsy sampling or the electrode placement during an operation.

5. The venous interventional integrated device for the brain tissue biopsy or the brain-computer interface electrode placement via the cerebral surface veins according to claim 1, wherein, except for the balloon inflation channel being connected to the balloon lumen, the guide channel and working channel are isolated from the balloon lumen.

6. The venous interventional integrated device for the brain tissue biopsy or the brain-computer interface electrode placement via the cerebral surface veins according to claim 1, wherein the balloon inflation channel, the guide channel, and the working channel are integrated into a single catheter, with each channel separated at the proximal end of the catheter for connecting to a contrast agent injection device, threading the micro-guidewire, and threading the puncture needle, respectively.

7. The venous interventional integrated device for the brain tissue biopsy or the brain-computer interface electrode placement via the cerebral surface veins according to claim 1, wherein the distal end and the proximal end of the balloon and an exit end of the working channel of the catheter are each provided with metal markers to facilitate positioning of the balloon and an exit position of the puncture needle during an operation.

8. The venous interventional integrated device for the brain tissue biopsy or the brain-computer interface electrode placement via the cerebral surface veins according to claim 1, wherein the venous interventional integrated device is equipped with a sealing and repair wire, the sealing and repair wire is allowed to be delivered through the working channel, a front end of the sealing and repair wire is provided with a sealing head, wherein the sealing head is severable, a front end of the sealing head is provided with a self-expandable umbrella-shaped biological membrane made of an absorbable material; when the sealing and repair wire is delivered through the working channel, the umbrella-shaped biological membrane is constrained by the working channel and folded around a periphery of the sealing head, and when the umbrella-shaped biological membrane exits the working channel, the umbrella-shaped biological membrane automatically expands forward and outward.

9. The venous interventional integrated device for the brain tissue biopsy or the brain-computer interface electrode placement via the cerebral surface veins according to claim 1, wherein the venous interventional integrated device is equipped with a sealing and repair wire, the sealing and repair wire is allowed to be delivered through the working channel, a front end of the sealing and repair wire is provided with a sealing head, wherein the sealing head is severable, a front end and a rear end of the sealing head are provided with two umbrella-shaped biological membranes made of an absorbable material, wherein the two umbrella-shaped biological membranes are self-expandable and arranged in opposite directions; when the sealing and repair wire is delivered through the working channel, the two umbrella-shaped biological membranes are constrained by the working channel and folded around the front end and the rear end of the sealing head, and when a front umbrella-shaped biological membrane exits the working channel, the front umbrella-shaped biological membrane automatically expands forward and outward, and when a rear umbrella-shaped biological membrane exits the working channel, the rear umbrella-shaped biological membrane automatically expands backward and outward.

10. The venous interventional integrated device for the brain tissue biopsy or the brain-computer interface electrode placement via the cerebral surface veins according to claim 1, wherein the working channel is further configured to deliver a venous wall repair material to seal and repair a puncture site, the venous wall repair material is made of a biological material, wherein the biological material is adhesive to a venous wall and allowed to be absorbed by the venous wall.
